(19) United States
(12) Patent Application Publication
(10) Pub. No.: US 2023/0233871 A1
(43) Pub. Date: Jul. 27, 2023

(54) ADAPTIVE DERMAL CARE METHODS AND APPARATUS

(71) Applicant: Cosmetic Edge Pty Ltd, Kew (AU)

(72) Inventor: Mathew JAFARZADEH, Kew, Victoria (AU)

(21) Appl. No.: 18/007,968

(22) PCT Filed: Jun. 1, 2021
(86) PCT No.: PCT/AU2021/050538

§ 371 Date: Dec. 2, 2022

(30) Foreign Application Priority Data
Jun. 3, 2020 (AU) 2020901818

(51) Int. Cl. A61N 5/06; A61N 1/32; A61H 23/02; A61N 1/40; A61M 35/00; A61N 1/08

(52) U.S. Cl. CPC A61N 5/0616; A61N 1/325; A61H 23/0236; A61N 1/403; A61M 35/003; A61H 23/0254; A61N 1/08; A61N 2005/0663; A61H 2201/0207; A61H 2201/5082; A61H 2201/5058; A61H 2201/105; A61H 2201/10; A61H 2201/5092; A61H 2201/5002; A61N 2005/0626; A61N 2005/0652; A61H 2201/503; A61H 2201/0107; A61H 2201/123; A61H 2201/0221; A61M 2205/054; A61M 2205/051; A61M 2205/05; A61H 2205/022; A61H 2201/5043; A61H 2201/0153; A61M 2205/3368; A61N 2005/0644

(57) ABSTRACT

The invention is broadly directed to a skin care apparatus comprising: a camera; a light device for emitting at least one of a blue, a green, a yellow and a red light; a vibration pad; a heating element; a heat sensor; a skin moisture sensor; an iontophoresis module; and a control processor configured to: operate the camera and the skin moisture sensor to perform a skin quality assessment of a user's skin, and operate any one or more of: the light device, the vibration pad, the heating element, the heat sensor, and the iontophoresis module to provide a bespoke skin treatment for the user's skin based on the skin quality assessment.

### TECHNICAL FIELD

The invention is directed to a skin care apparatus configured to provide a plurality of skin treatments to the skin of a user. The invention further provides a skin care apparatus configured to perform a skin assessment of a user's skin and provide a skin treatment based on the skin assessment.

### BACKGROUND

Known dermal care products advertise a particular solution and expected (glorified) result. Tens to hundreds of facial or skin creams and lotions are marketed as being able to reduce wrinkles, blemishes, lesions, or acne (or the appearance of wrinkles, blemishes, lesions or acne). Indeed, an entire aisle in a pharmacy contains such products, in addition to the constant barrage of television and media advertising these products.

For any of the above mentioned products, the instructions for use are generic. For example, facial creams may provide general instructions for daily or twice daily use. Skin care devices may have instructions that provide a usage time duration and an explanation of different settings. Oftentimes, the user has to experiment as to the best use of the products to achieve the desired skin care results. However, in many instances, users never achieve optimal, or even any noticeable results and become discouraged using the skin care products—often to seek out other products. In other instances, a user may believe a suboptimal result is the best available result, and continue using the skin care products not realizing more optimal results are possible.

A small number of people may consult with dermatologists to improve skin health and appearance. However, even for these people, skin care can only improve with continued treatment, which may not be financially possible or fit within someone's schedule. As a result of all of the above issues, current skin care products and solutions are not satisfactory for many people.

The present invention was conceived with these shortcomings in mind.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

### SUMMARY

The example methods and apparatus disclose herein an adaptive dermal care solution. In an example, a skin care device is communicatively coupled to a smartphone or tablet computer. An application on the smartphone or tablet computer provides control for the skin care device to provide an assessment of a user's skin quality. The skin care device may include one or more sensors or cameras for measuring properties of a user's skin including moisture level, pH, collagen level, pigmentation level, etc. The skin care device may also provide measurements for lesions. Measurements recorded by the skin care device are transmitted to the application, which processes the measurements to provide graphical and/or textural skin quality information to the user.

In a first aspect, the invention provides a skin care apparatus comprising: a camera; a light device for emitting at least one of blue, green, yellow and red light; a vibration pad; a heating element; a heat sensor; a skin moisture sensor; an iontophoresis module; and a control processor configured to: operate the camera and the skin moisture sensor to perform a skin quality assessment of a user's skin, and operate any one of more of: the light device, the vibration pad, the heating element, the heat sensor, and the iontophoresis module to provide a bespoke skin treatment for the user's skin based on the skin quality assessment.

In some embodiments, the skin care device may provide one or more skin care treatment recommendations based on the determined skin quality of the user. For example, the application of provide recommendations (and corresponding operating procedures) to address skin aging, acne, skin redness, and/or skin sensitivity. Further, the application may provide recommendations for using a cleanser, toner, moisturizer, astringent, facial mask, and/or exfoliating agent. The application may communicate with the skin care device to track when certain skin care therapies are performed as feedback to determine if one or more recommendations are to be modified. Further, the application may track skin quality over time and use the change over time in conjunction with known therapies performed to determine if the recommendations are to be modified. In other words, the application is configured to converge upon an optimal skin care solution that is bespoke for the user.

As disclosed in more detail below, the example skin care device includes a camera, a skin heat sensor, and skin moisture sensors. The example skin care device also includes a camera light/flash, one or more LEDs providing light around wavelengths of 455 nm, 550 nm, 580 nm, 633 nm, and 830 nm. The example skin care device further includes a vibration pad, a heating element to generate surface heat, and an iontophoresis module. Further, in some embodiments, the skin care device may include or be connectable to a fluid container for controlled dispensing of a skin care-related fluid. In these embodiments, the skin care device may include one or more apertures or pores for dispensing the fluid and a pressure sensor to sense contact with a user's skin for causing the fluid to be dispensed.

A use a multi-wavelength LED rings of 450 nm-825 nm (red, infrared, green , blue and yellow) placed at the top of the device head, are placed for treatment of variety of skin conditions such as rejuvenation, collagen stimulation, acne therapy, sensitivity and stimulate healing due to various injuries and inflammation.

In some embodiments, the example skin care device disclosed herein includes or is connected to a plasma jet. The disclosed plasma jet is configured to generate plasma for locally heating a user's skin. The application on the user device may be configured to control a skin penetration depth and/or control activation by detecting when the plasma jet is aligned with a skin lesion.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

In some embodiments, the skin care apparatus may further comprise a transceiver for communication with an application on a user device, wherein the control processor is configured to operate in connection with the transceiver to: transmit an image from the camera and data from the skin moisture sensor to the application for performing the skin quality assessment; and receive instructions from the application for operating any one or more of the light device, the vibration pad, the heating element, the heat sensor, and the iontophoresis module to provide the skin treatment.

The application may be configured to use the image from the camera and the data from the skin moisture sensor to determine skin quality parameters for the user's skin including at least one of a collagen level, a pH level, a moisture/dryness level, a detection of wrinkles/lesions, or a detection of acne. The application may be configured to determine and display a recommendation based on the skin quality parameters, the recommendation including at least one cream or fluid type.

In some embodiments, the application may be configured to determine the instructions for operating the light device, the vibration pad, the heating element, the heat sensor, and the iontophoresis module based on at least one of the recommendation or the skin quality parameters. The recommendation may be selectable and selection of the recommendation causes the application to transmit the instructions to the control processor. The selection of the recommendation may cause a schedule of treatments to be created in a calendar on the skin care apparatus.

In some embodiments, the application may be configured to specify in the instructions that a red LED of the light device is to be activated for up to five minutes per treatment for skin quality parameters corresponding to an anti-aging treatment or a skin sensitivity treatment. The application may be configured to specify in the instructions that a blue LED of the light device is to be activated for three to five minutes and that an acne control fluid is to be dispensed per treatment for skin quality parameters corresponding to an acne control treatment.

The light device may include at least one LED that emits blue light at about 455 nm, at least one LED that emits green light at about 550 nm, at least one LED that emits red light at 633 nm, at least one LED that emits yellow lights at about 580 nm and at least one LED that emits infrared light at 830 nm to promote skin healing.

In some embodiments, the vibration pad may include a vibration actuator configured to vibrate at a frequency to numb the user's skin during the skin treatment. The vibration actuator may be configured to vibrate at 7000 RPM to provide acoustic waves at the frequency to numb the user's skin.

In some embodiments, the heating element includes a radio transmitter configured to provide localised tissue heating via radiofrequencies between 100 kHz-300 GHz. The iontophoresis module may be configured to provide a voltage gradient to the user's skin through creation of an electric field. The iontophoresis module may comprise at least one galvanic plate to deliver a current or a voltage to the skin of a user when applied thereto.

In some embodiments, the apparatus may comprise a fluid dispensing system for dispensing a fluid, the fluid dispensing system including: a fluid container comprising at least one cream, lotion, or fluid for the skin treatment; a pump or actuator to drive the at least one cream, lotion, or fluid from the fluid container; and an outlet to dispense the at least one cream, lotion, or fluid to the user's skin. The fluid container may be deformable. The fluid container may be rigid. The fluid container may comprise a single compartment for storing fluid. The fluid container may comprise a plurality of compartments for storing a plurality fluids. Selected fluids of the plurality of fluids may be dispensed in combination. Alternatively, individual fluids may be selectively dispensed in isolation from other fluids of the plurality of fluids. In some embodiments, the fluid containers may be recyclable and in still further embodiments, the fluid containers may be refillable from a fluid store. The fluid container may work in combination with a valve to control the dispense rate of fluid therefrom. The valve may be a duck bill valve. The pump may be a peristaltic pump that draws fluid from the fluid container. In some embodiments the actuator may be a plunger that deforms a portion of the fluid container to urge fluid therefrom. In some embodiments the actuator may be a roller or a pair of rollers that exert a force onto the container to drive fluid from the fluid container.

The fluid container may be removed and replaced with an alternate container containing an alternative type of cream or serum via an inserting and removing action or by pressing a release button of the device to load and unload the fluid container.

The pumping action of the fluid container may be a peristaltic, an air pumping or a spinning motor type where fluid is pushed and ejected gradually in accordance with a preselected amount.

In some embodiments, the at least one of the iontophoresis module, the vibration pad, or the heating element may be operated in conjunction with dispensing the at least one cream, lotion, or fluid to the user's skin to promote transdermal delivery. The fluid container may be removable and selected from a plurality of fluid containers based on a type of the skin treatment. The skin treatment may be selected from any one of more of an anti-aging treatment, a hydrating treatment, a sensitivity treatment, a pre-treatment, a moisturising treatment, a healing treatment, an acne treatment, a scar reduction treatments and the like.

In some embodiment, the apparatus may further comprise a user interface including a sense button, wherein activation of the sense button causes the skin moisture sensor to detect data including at least one of a skin moisture level, a skin dryness, and/or a pH level of the user's skin. In some embodiment, the apparatus may further comprise a user interface including a capture button, wherein activation of the capture button causes the camera to record an image of the user's skin. The user interface may be an integrated display on the apparatus. In some embodiments, the user interface may be displayed on a paired electronic device, for example a smart phone or computer that can receive, and store data from each skin quality assessment conducted by the apparatus.

### BRIEF DESCRIPTION OF THE FIGURES

Exemplary embodiments of the invention are described below by way of example only, and not by way of limitation. Referring now to the accompanying drawings in which like numerals indicate like elements throughout the several figures:

FIG. 1 shows a diagram of a skin care environment including a skin care device or apparatus and an application operating on a user device, according to an example embodiment of the present disclosure.

FIG. 2 is a diagram of a fluid container that connects to the skin care device or apparatus of FIG. 1, according to an example embodiment of the present disclose.

FIG. 3 is a diagram of an application face disposed on a contact surface of the skin care device or apparatus of FIG. 1, according to an example embodiment of the present disclosure.

FIGS. 4 to 9 are diagrams showing a skin quality assessment performed by an application on the user device of FIG. 1, according to example embodiments of the present disclosure.

FIGS. 10 and 11 are images of a skin care device or apparatus, according to an embodiment of the present disclosure, the device docking with a recharging base to recharge an internal power source of the device.

FIGS. 12A-12F illustrate different contemplated methods of dispensing fluid from fluid container within the skin care device.

FIGS. 13A-13C illustrate diagrams of different configurations of a skin care devices according to various embodiments of the invention.

FIGS. 14A-14C illustrate packaging layouts of internal componentry within the configurations of the skin care devices illustrated in FIGS. 13A-13C.

FIGS. 15A and 15B illustrate a top perspective view and a rear view of a skin care device according to an embodiment of the invention.

FIG. 16 is an exploded view of the skin care device of FIGS. 15A-15B illustrating the internal layout of components therein.

FIG. 17 is an exploded view of a replaceable fluid delivery pod for use with the skin care device of FIGS. 15A-15B.

FIGS. 18A-18D illustrate bottom, top perspective, bottom perspective and top views of the pod of FIG. 17 respectively.

FIGS. 19A and 19B are bottom and top perspective views of a skin care device according to a further embodiment of the invention.

FIGS. 20A-20D illustrate top perspective, open-side, bottom perspective and open-top perspective views of the skin care device of FIGS. 19A and 19B, respectively.

FIG. 21 is an exploded view of the skin care device of FIGS. 19A-19B illustrating the internal layout of components therein.

FIGS. 22A-22D illustrate top perspective, bottom, bottom perspective and top views of the pod of FIG. 21, respectively.

FIGS. 23A 23B illustrated two cross-sections views of the pod of FIG. 17 illustrating by arrow a direction of movement of a pod evacuator or diaphragm that forces the fluid out of the pod on demand

FIG. 24 is an exemplary user interface that can be available from on a display of the device itself, or a display of a paired electrical device, illustrating available features and skin analysis results to the user.

Embodiments will now be described more fully hereinafter with reference to the accompanying drawings, in which various embodiments, although not the only possible embodiments, of the invention are shown. The invention may be embodied in many different forms and should not be construed as being limited to the embodiments described below.

### DETAILED DESCRIPTION

The present disclosure relates in general to a method and apparatus for adaptive skin care. The disclosed method and apparatus are configured to detect a user's skin quality using one or more sensors. Skin quality measurements are used for providing treatment or therapy recommendations. Overtime, the recommendations may be adjusted by tracking how the user's skin quality has changed in relation to the applied therapies or treatments. An algorithm of the application on the user device is configured to converge upon an optimal skin care solution for a user.

Throughout the disclosure, reference is made to user devices. As disclosed herein, a user device can include any cellphone, smartphone, personal digital assistant (“PDA”), mobile device, tablet computer, computer, laptop, server, processor, console, gaming system, multimedia receiver, workstation, disk storage device, or any other computing device.

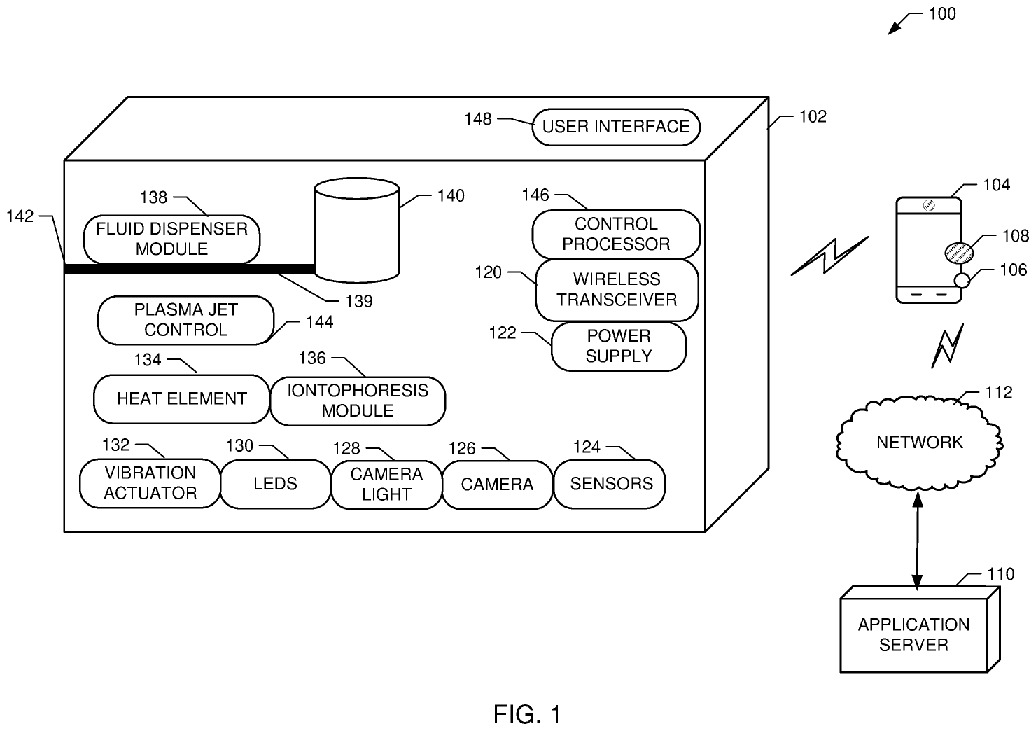

Reference is also made herein to facial skin care. It should be appreciated that the example method and apparatus may be applied to other skin areas, such as a user's neck, arms, hands, legs, feet, buttocks, chest, under-arm area, back etc. FIG. 1 is a diagram of an example skin care environment 100 including a skin care apparatus or device 102 and a user device 104. The example user device 104 includes a processor 106 that is configured to execute a skin care application 108. The application is defined or specified by one or more instructions stored in a memory of the user device 104. Execution of the instructions by the processor 106 cause the processor 106 to perform the operations described herein in connection with the application 108.

The example user device 104 is configured to communicatively couple to the skin care device 102 via a wired or a wireless connection. A wired connection may include a USB connection, a Lightning® connection, etc. A wireless connection may include a Bluetooth® connection, a Zigbee® connection, an NFC connection, a Wi-Fi connection, etc.

In some embodiments, the application 108 may be communicatively coupled to an application server 110 via a network 112 (e.g., any wide area network, cellular network, or combinations thereof). The application server 110 may be configured to store template recommendations, therapies, and/or treatments for access by the application 108. Such recommendations may be newly created as additional uses of the device 102 are determined/approved. Further, the recommendations may be modified overtime based on feedback from a population of users. The application 108 is configured to download the recommendations for customization to a user's skin quality and/or past treatment history.

In some embodiments, the application 108 is configured to transmit measurement and/or user treatment data to the application server 110. The application server 110 analyses the data and can provide one or more recommendations for the user. In some instances, the application server 110 can include the data in machine modelling algorithms for refining the skin care recommendation templates. Further, in some embodiments, the application server 110 can make a user's information available to a skin care professional for consultation.

The example skin care device 102 includes a wireless (or wired) transceiver 120 for communication with the user device 104. In some embodiments, the transceiver 120 can provide for pairing with the user device 104. The example skin care device 102 also includes a power supply 122. The example power supply 122 may include one or more batteries 159 and/or a connection to a power outlet. The power supply 122 provides power for the skin care device 102. The batteries 159 can be rechargeable and the device 102 can be configured to dock with a charging base 169 when not in use, to replenish charge in the batteries 159.

The example skin care device 102 can also include one or more sensors 124. The sensors 124 can include a skin heat sensor, a skin moisture sensor, and a pH sensor for measuring parameters of skin quality. In some embodiments, the sensors 124 can also include a pressure sensor, a biometric sensor, proximity, and/or conductivity to detect when the skin care device 102 is placed adjacent or in contact with a user's skin.

The example skin care device 102 can also include a camera 126, such as a high-resolution camera, an ultraviolet (“UV”) camera, and/or an infrared camera, and a camera light 128. The example camera 126 and camera light 128 are configured to operate in cooperation to record images of a user's skin, including lesions, pigments, moles, hair, scars, etc. In addition to the camera 126, the skin car device 102 can also include a magnifying glass to provide for manual user inspection of their skin.

The example skin care device 102 can further include one or more LEDs 130. The example LEDs are configured to emit light at wavelengths that promote skin healing. For example, the LEDs 130 may include LEDs that emit light around 455 nm, light around 633 nm, and/or light around 830 nm. It should be appreciated that the LEDs 130 may emit light at additional or fewer wavelengths.

The example skin care device 102 can moreover include a vibration actuator 132, which can include a vibrating pad. As disclosed herein, the vibration actuator 132 can vibrate at one or more frequencies as part of a skin quality assessment and/or a skin treatment. In some instances, the vibration actuator 132 can vibrate at a frequency configured to numb a user's skin during treatment. In some embodiments, the vibration actuator 132 can be configured to provide vibration via acoustic waves. The actuator 132 can operate at 7,000 RPM to generate acoustic waves for inducing tissue vibration.

To apply skin surface heat, the skin care device 102 includes one or more heat elements 134. The example heat element 134 is configured to radiate heat up to a temperature between 40° C. and 50° C. In other embodiments, the heat element 134 includes a radio transmitter configured to provide localised tissue heating via radiofrequencies. The element 134 can provide heating using a frequency between 100 kHz-300 GHz.

The example skin care device 102 can also include an iontophoresis module 136, which applies a voltage gradient on a user's skin through the creation of an electric field. The iontophoresis module 136 can be used for transdermal delivery to a user's skin of a fluid 101 provided in a fluid container 140. The use of fluid 101 facilitates ionized (charged) particles to traverse the natural barrier provided by the user's skin to thereby enhance absorption and delivery of molecules in the fluid 101 into the skin. In some embodiments the fluid 101 can be a medicament, or an alternative skin treatment fluid, in other embodiments the fluid 101 is a light serum, while in other forms the fluid 101 is a heavier cream or lotion. In these examples, the iontophoresis module 136 is used in conjunction with a fluid dispenser module 138, which controls dispensing of the fluid 101 from the fluid container 140.

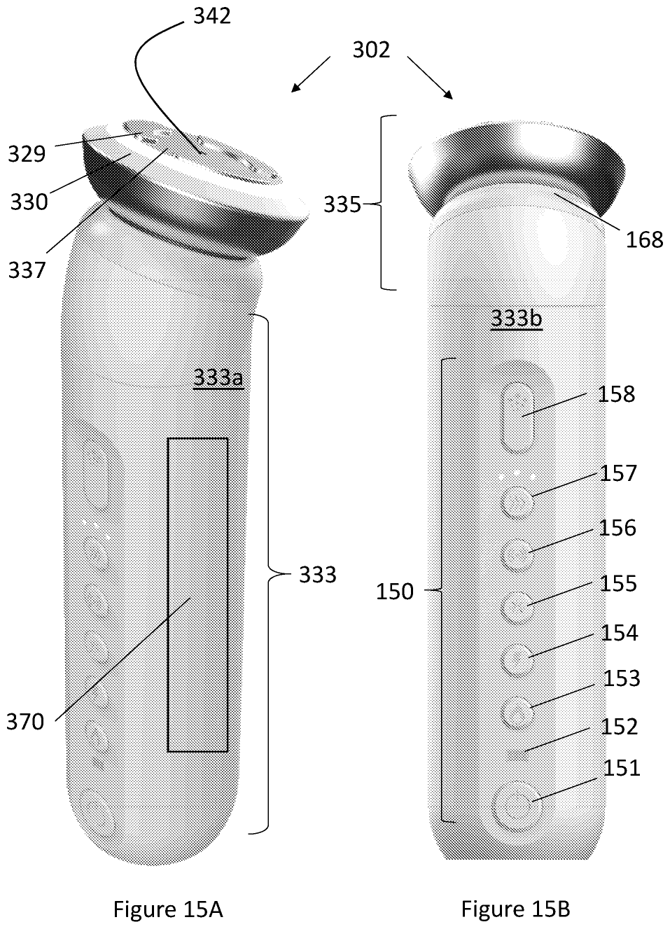

In other embodiments, a user can first apply a cream or lotion to their skin, such that subsequent use of the iontophoresis module 136 of the skin care device 102 can promote transdermal propagation of the fluid (cream or lotion) across one or more layers of skin. The iontophoresis module 136 can comprise one or a plurality of galvanic plates (129) located about the device 102 and configured for contact with skin of the user (described herein in reference to FIGS. 15-21).

The fluid 101 can be selected dependent on the type of treatment to be applied to the user's skin. After skin analysis the device 102 will provide information to the user as to whether a moisturising treatment, a healing treatment, an acne treatment, an aging treatment or scar reduction treatments is recommended. The user can then select the appropriate fluid to be dispensed by the device 102 for the selected treatment

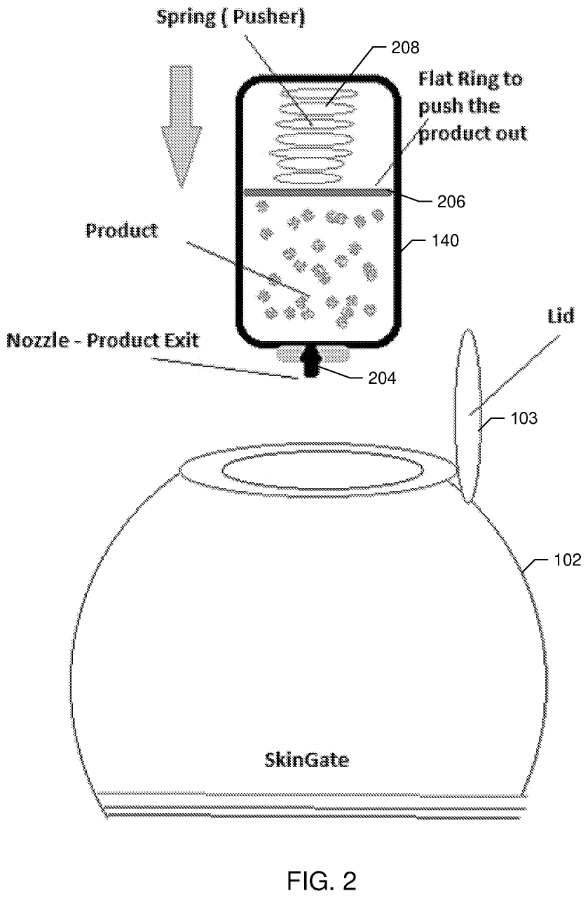

The fluid dispenser module 138 is configured to control one or more valves or actuators for moving a fluid from the fluid container 140 to one or more apertures or pores 142 via a fluid conduit 139 for dispensing the fluid 101. FIG. 2 is a diagram of the fluid container 140, according to an embodiment of the present disclosure. In the illustrated embodiment, the fluid container 140 is removably connected to the skin care device 102. The container can contain between 10 mL and 50 mL, preferably around 15 mL of fluid.

To insert or remove, a user opens a lid 103 on the device 102, which provides access to a connector (not shown). A nozzle 204 of the fluid container 140 is configured to engage with and form a fluid-tight connection with the connector of the device 102.

In some embodiments, the container 140 includes a partition 206 that defines a space for the fluid 101 (i.e., product). The partition 206 can include a flat ring or other head configured to provide even force against the fluid 101. An actuator 208, such as a spring, provides force on the partition 206, thereby pushing the partition 206 towards the nozzle 204 to cause fluid to be dispensed.

In an embodiment, a valve 141 in the nozzle 204 of the container 140 may enable fluid to exit at a certain pressure above a threshold. The force applied by the spring/actuator 208 can be below the threshold. The device 102 can include an actuator, such as controlled by the fluid dispenser module 138, to apply addition force to overcome the threshold, thereby enabling fluid 101 to be pushed from the container 140.

In other embodiments, connection of the nozzle 204 with the connector of the device 102 causes a seal in the nozzle to open or break, thereby enabling fluid 101 to be released only when the fluid container 140 is inserted (and can prevent fluid leakage when the container 140 is removed). The skin care device 102 can include a valve and/or a pump 145 (such as a rotary pump or peristaltic pump) that is controlled by the fluid dispenser module 138. Before use, the valve may be closed or the pump 145 placed in a position to occlude the fluid conduit 139 to prevent fluid leakage. When a control signal is received, the fluid dispenser module 138 is configured to open the valve and/or control a pumping speed to enable the fluid 101 to be transferred from the container 140 to the aperture 142 via the conduit 139.

Returning to FIG. 1, in some instances, the fluid dispenser module 138 can be controlled in cooperation with the iontophoresis module 136, the vibration actuator 132, the heat element 134, and/or the LEDs 130 for providing a comprehensive or complete skin treatment or therapy. The use of heat, iontophoresis, and vibration can promote skin absorption of the applied fluid.

In some embodiments, the skin care device 102 can include plasma jet control 144. In these embodiments, a small tip or other attachment can be connected to the device 102. The tip is controlled by the plasma jet control 144 to generate a plasma for inducing a sublimation process. The process is configured to heat the dermis in a targeted area, which causes peeling and shedding post treatment. The enables new, fresher skin and/or collagen to form.

Further, in some embodiments, the skin care device 102 can be configured with the components 120 to 138 and 144 described above and/or other components to provide for electroporation treatments, mesoporation treatments, iontophoresis treatments, or combinations thereof.

The example components 120 to 138 and 144 can be controlled by a control processor 146. The example control processor 146 is configured to send activation signals to each of the components 120 to 138 and 144 when activation is needed. For example, during a skin assessment routine, the control processor 146 transmits activation signals to the sensors 124, the camera 126, the camera light 128, and/or the LEDs 130 to record measurements and/or images of skin quality parameters. These measurements and/or images can include timing sequences of the camera light 128 and camera 126 to record one or more images, and/or controlling sequences of the LEDs 130 for recording one or more images. Images and/or measurement data from the sensors 124 and 126 are received in the control processor 146, which can be transmitted to the application 108 on the user device 104.

The example control processor 146 is also configured to transmit signals for activation of the heat element 134, the vibration actuator 132, iontophoresis module 136, the LEDs 130, the fluid dispenser module 138, and/or the plasma jet control 144 to perform one or more therapies or treatments. In some embodiments, a therapy or treatment is received from the application 108, and specifies a routine that defines which components are to be activities for certain durations. The routine can also specify an intensity, frequency, and/or duration for activation. The example control processor 146 is configured to provide control instructions based on the routine.

In an example, the control processor 146 can receive a message from the application 108 including a routine. The routine can specify in a first phase that a user's skin is to be heated to 40° C. During this first phase, the control processor 146 transmits control instructions to the heat element 134, and uses heat sensor 124 feedback to ensure the user's skin is heated to the specified temperature. In a second phase, the routine can specify a frequency, amplitude, and wave pattern for the vibration actuator 132, a volume of fluid to be dispensed by the fluid dispense module 138, and an amount of iontophoresis to be applied by the module 136. In response to the routine, the control processor 146 transmits corresponding signals to activate the components 132, 138, and 136 as specified. Finally, in a third phase, the routine can specify recording images of the result of the skin treatment using the camera 126, which cause the control processor 146 to acquire the specified image. In some embodiments, the control processor 146 may transmit status information to the application 108 to display, for example, operations being performed during each phase of a treatment and/or parameters of the different components being activated.

The example skin care device 102 of FIG. 1 can also include a user interface 148. In some embodiments, the interface 148 can include one or more buttons 150 or switches, such as a power button 151, a communication pairing button, a record button for manual activation of the camera 126, a light button 155 for manual activation of the camera light 128 and/or LEDs 130, a dispense button 158 for manual dispensing of fluid from the container 140, and/or a start/pause/stop control for beginning, pausing, and stopping a treatment or therapy.

In some embodiments, the user interface 148 may include a “sense” button, selection of which causes the control processor 146 to record measurements of skin moisture, dryness, and/or pH level, which are transmitted to the application 108 for storage/tracking/display. Further, the user interface 148 may include a “capture” button, selection of which causes the control processor 146 to record one or more photos of a user's skin, which are transmitted to the application 108 for storage/tracking/display. The user interface 148 can also include an “infuse” button 158, selection of which causes the components 138, 136, 134, 132, and/or 130 to operate in cooperation as controlled by the control processor 146 to perform a skin treatment.

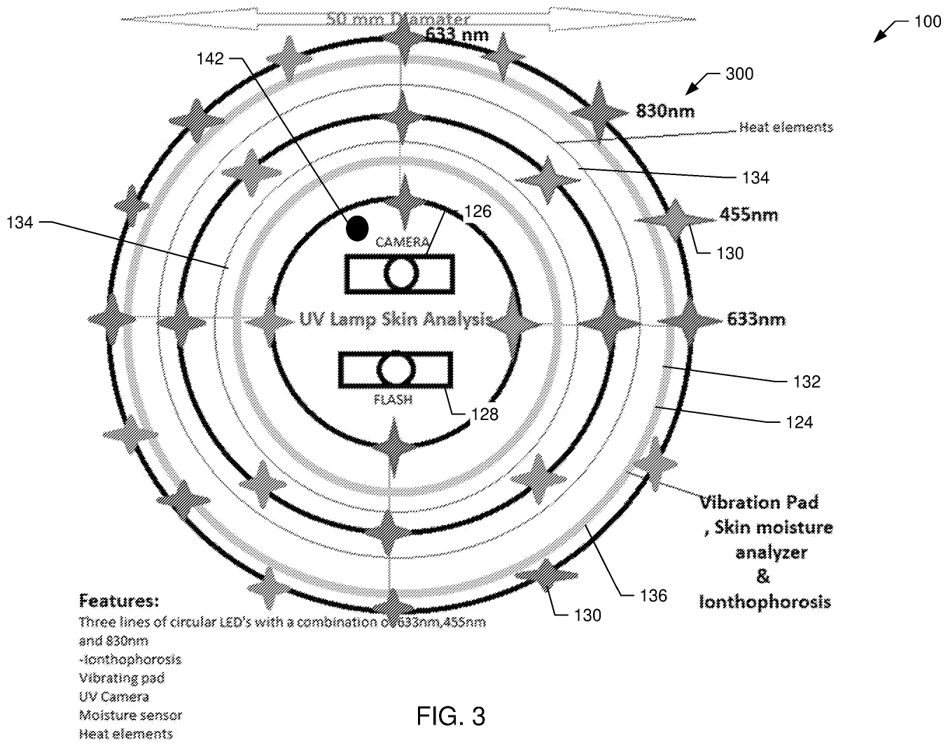

FIG. 3 is a diagram of an application face 300 of the skin care device 102 of FIG. 1, according to an example embodiment of the present disclosure. The application face 300 is configured to contact a user's skin for skin quality assessment and/or treatment. As shown, the application face 300 includes three concentric circles of LEDs 130. The LEDs 130 have alternating transmission wavelengths of 455 nm, 633 nm, and 830 nm. It should be appreciated that LEDs with different wavelengths may be used and/or the LEDs may be provided in alternative arrangements, such as in an oval or grid pattern.

The application face 300 also includes the camera 126 and the camera light 128 for recording images. Further, the application face 300 includes a vibration pad for operation with the vibration actuator 132, heat elements 134 and an iontophoresis applicator for operation with the iontophoresis module 136. The application face 300 also includes moisture, pH, etc. sensors 124 and at least one aperture or pore 142 for dispensing fluid 101 from the container 140 that is connected to the skin care device 102.

In some embodiments, the application face 300 may further include an attachment for a tip for operation with the plasma jet control 144. In other embodiments, the attachment for the tip may be located on a side of the skin care device 102 rather than on the application face 300. It should be appreciated that the components shown on the application face 300 are illustrative only and that the application face 300 may include additional or fewer components.

### Skin Care Application Embodiments

As described above, the skin care application 108 operates on the user device 104 for assessing properties of a user's skin quality and applying one or more treatments. The example application 108 is configured to receive, from the skin care device 102, sensed biological characteristics of a user's skin. The application 108 is configured to analyse the biological characteristics to determine a diagnosis. Based on the diagnosis, the application is configured to recommend at least one skincare or therapeutic treatment/solution to address identified skin issues. The application 108 can also be configured to launch a web browser with a webpage of recommended skincare products for purchase and/or contact information for a therapist/specialist.

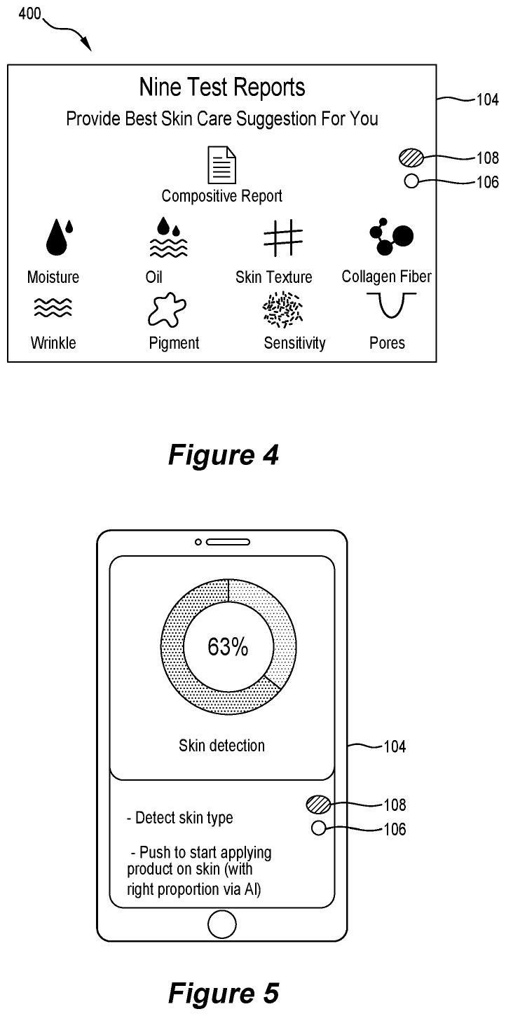

FIGS. 4 to 8 are diagrams showing a skin quality assessment performed by the application 108, according to example embodiments of the present disclosure. FIG. 4 shows a dashboard interface 400 with an option for a user to determine their skin quality (e.g., moisture level, oil, skin texture, collagen fibre, wrinkles, pigment, sensitivity, and pores) using the skin care device 102. The dashboard interface 400 may also display usage history, progression/tracking analysis, and/or provide a library of treatment images overtime.

Selection of the “Detect Skin” option causes the application 108 to open analysis interface 500 of FIG. 5. The interface 500 can have a progress bar of the detection routine being operated by the skin care device 102. In some embodiments, the interface 500 may indicate which analysis is currently being performed. User interface 600 shows results of the analysis. The results can include an indication of a skin moisture level, a pH level, skin pigment information, collagen level, etc. In some embodiments, the application 108 can perform image analysis to identify wrinkles, blemishes, scars, acne, moles, etc. The application 108 can perform template matching to templates of known wrinkles, blemishes, scars, acne, moles, etc. In other embodiments, the application 108 can determine dimensions, shape, and/or depth of the wrinkles, blemishes, scars, acne, moles, etc. and use a machine learning model for identification.

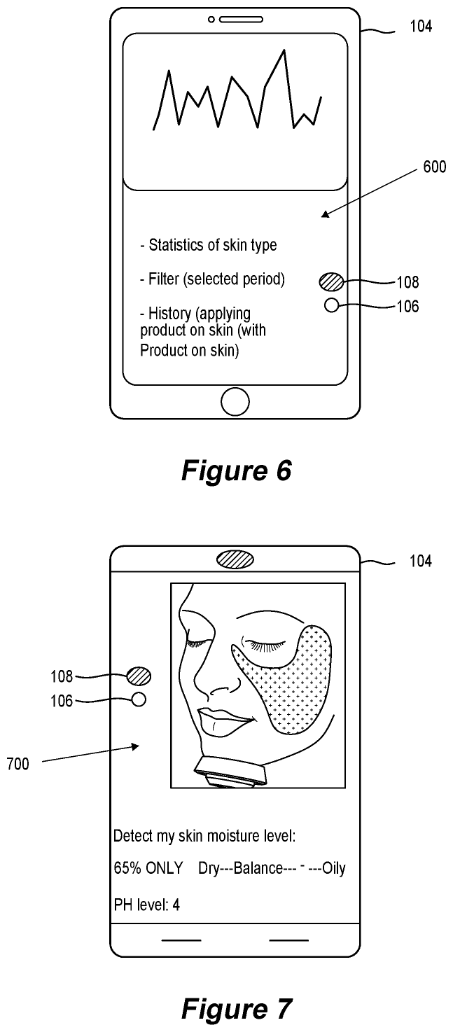

Also, as shown in FIG. 6, the user interface 600, the application 108 is configured to track a user's skin quality overtime. This can include changes to moisture level, pH, wrinkle depth/size, deformation size, etc. Further application 108 can store time/date stamped images to show the skin change over time.

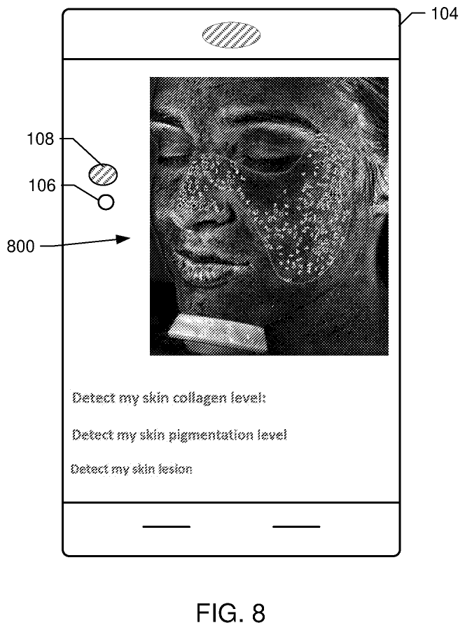

FIG. 7 shows a user interface 700 with an image of a user's face. The application 108 superimposes skin moisture data over the image to areas that are dry, balanced, oily, etc. In some embodiments, the images are shown from a perspective the camera 126 placed adjacent or in contact with a user's skin. These images can direct the user to problem skin sites when using the device 102. FIG. 8 shows a diagram of a user interface 800 displaying skin collagen levels, which are superimposed over a grey-scale image. For detection of skin lesions, the application 108 can create a graphical circle or other highlight indicator around the identified lesion for superimposing over an image.

In some embodiments, the application 108 provides one or more skin treatment recommendations based on the skin quality assessment. The recommendations can be based on collagen levels, pH levels, moisture/dryness levels, detection of wrinkles/lesions, etc. In some instances, the application 108 may recommend use of specific creams or fluids as part of the treatment, such as creams for balancing moisture or pH based on the detected levels.

The application 108 can enable the recommendations to be selectable. After receiving a selection, the application 108 transmits a corresponding routine to the control processor 146. After selection of a start or similar button the user interface 148 (indicating that the user has applied any goggles and positioned the device 102 at the specified skin care area, the device 102 may provide a countdown and subsequently begin treatment.

For anti-aging and/or skin sensitivity, the routine can specify that red LEDs 130 are to be activated for three minutes. For acne control, the routine can specify that blue LEDs 130 are to be activated for three minutes. After or during, the fluid dispenser module 138 dispenses an acne control fluid. The application 108 can then provide periodic reminders for subsequent acne treatments or application of the acne control lotion. For oily skin, the application 108 may specify that fluid for oily skin should be used and inserted into the device 102. The device 102 may then dispense the fluid. Other routines can be available for cleanser application, toner application, moisturizer application, use of an astringent, exfoliation application, and use of a facial mask. Fur infusion of specified fluids 101, the routine can specify control of the heat element 134, the iontophoresis module 136, the vibration actuator 132, the fluid dispenser module 138, and/or the LEDs 130.

In some embodiments, the application 108 is configured to use tacking or feedback skin quality data for optimising or further refining the routines/recommendations. For example, an overcorrection of oily skin can occur overtime, where then the application 108 detects dry skin and applies the appropriate routine. In another example, lesions can be healed or otherwise hidden, thereby eliminating the need for such treatment. The example application 108 accordingly operates with the skin care device 102 to provide an optimised bespoke skin care solution for each user.

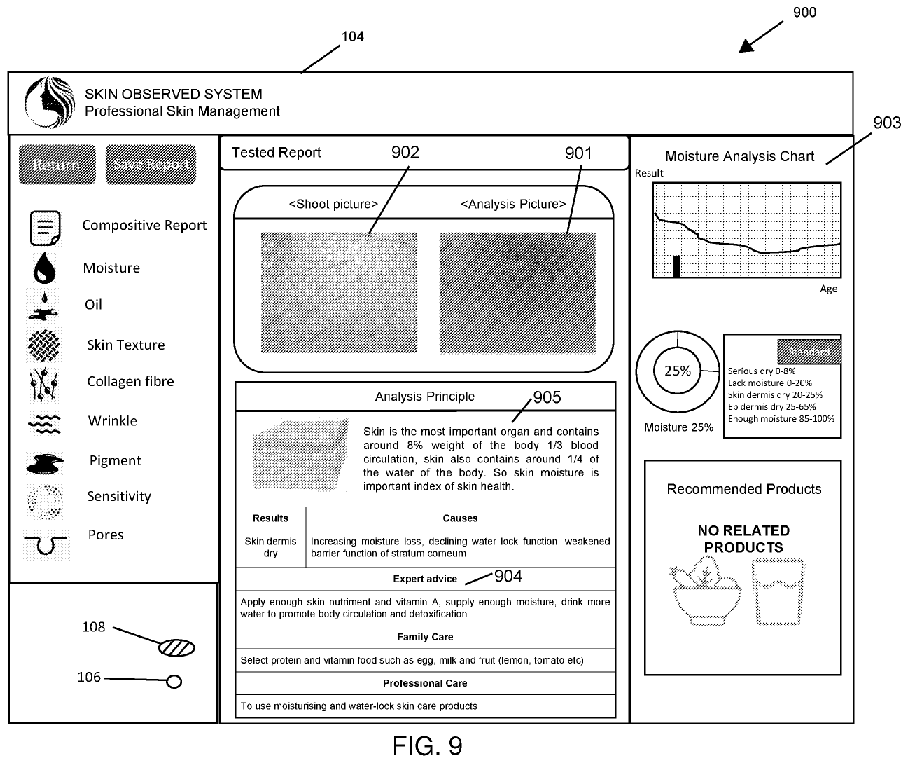

FIG. 9 is a diagram of a skin quality report 900 generated by the application 108 on the user device 104, according to an example embodiment of the present disclosure. The skin quality report 900 includes images of a user's skin recorded at different wavelengths of light 901, 902. The report 900 also includes information regarding skin moisture level in the form of a moisture analysis chart 903 and related information, possible causes for low moisture level, and recommendations for improving skin quality. Additionally, the report 900 can provide expert advice 904 and other useful information/recommendations to the user 905.

### Skin Care Device Embodiments

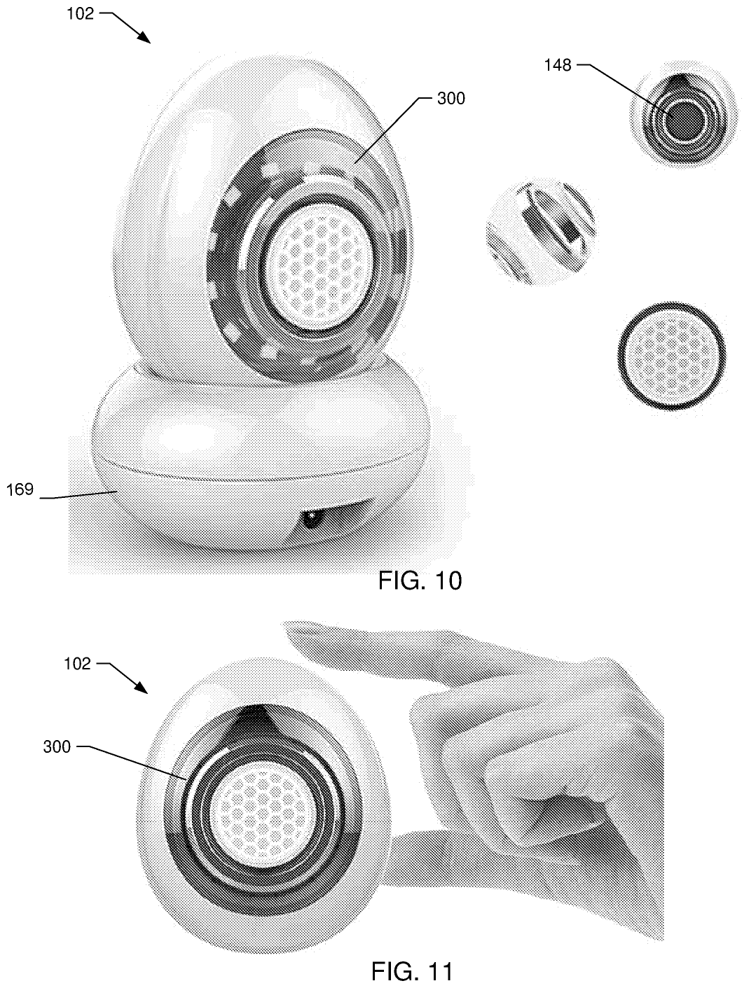

FIGS. 10 and 11 are diagrams of a first exemplary skin care device 102, according to an example embodiment of the present disclosure. In FIG. 10, LEDs of the application face 300 are illuminated. As shown, the skin care device 102 is hand portable. FIG. 10 also shows a reverse side of the skin care device 102 with the user interface 148. It should be appreciated that the skin care device 102 may have different form factors and/or sizes in other embodiments. The device 102 is illustrated on a charging base 169 providing cord free operation of the device 102.

A number of different fluids 101 can be used to treat skin, ranging from medicaments to creams and lotions. Medicaments can be selected to treat skin conditions of a medical nature while other fluids can improve texture, colour, ageing signs, scars and hydrate the user's skin. As this range of fluids 101 will have differing consistencies, viscosities and lifespans it is contemplated that a variety of different delivery methods can be employed by the device 102.

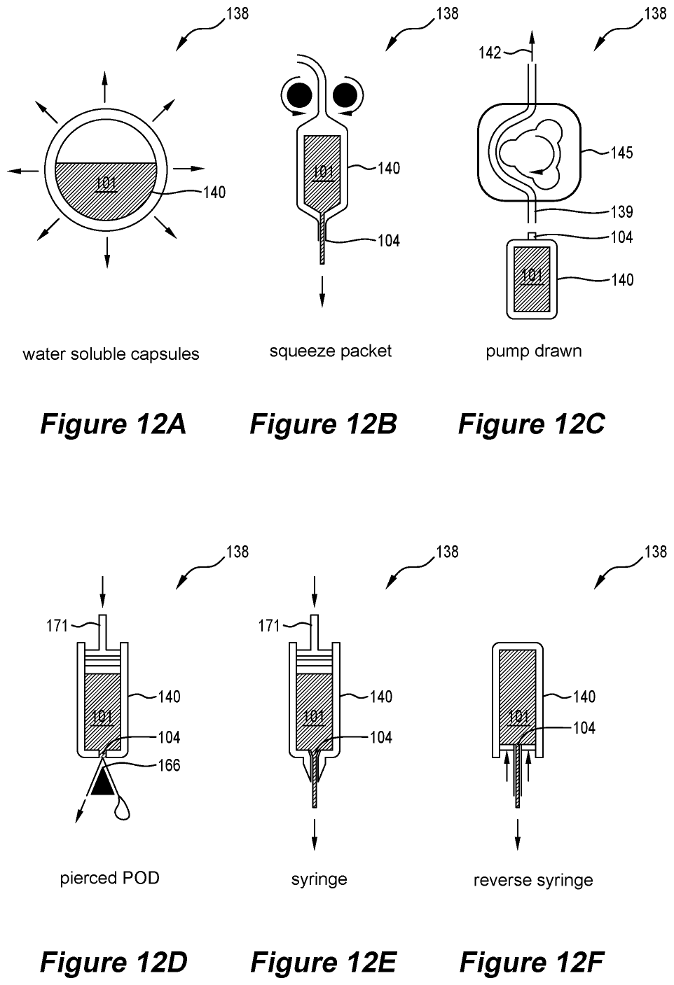

FIGS. 12A-12F illustrate a number of contemplated dispenser modules 138. Each is directed to a replaceable or reusable or refillable pod 140 that can be selected and located within the skin care device 102 for bespoke skin treatment plans. Not illustrated, but also contemplated are pods having more than one compartment for storing and dispensing more than one fluid selectively. A plunger mechanism 171 can be used to urge the fluid 101 from the pod 140, as such, the plunger mechanism 171 can be configured to urge the plurality of fluids in a compartmentalised pod 140 to selectively dispense a first fluid during treatment and a second fluid after treatment. Alternatively, the pod can contain a pre-treatment fluid, a treatment fluid and a post treatment fluid. In alternative embodiments, the skin care device 102 can be configured to receive a plurality of different pods 140, each tailored to a bespoke user's skin needs, for inserting into the device 102 for use during treatment.

FIG. 12A is a water soluble capsule that dissolves on contact with water, providing a minimal waste product that is flexible in shape.

FIG. 12B is a squeezable packet, that ejects fluid 101 when squeezed by roller from both sides or a single side when pressed against the skin care device 102. These packets are hygienic and flexible to fit a cavity within the device 102.

FIG. 12C illustrates a pod 140 connected via a conduit 139 to a pump 145. The pump 145 draws a selected amount of fluid 101 from the pod outlet orifice 104 on demand, drawing the fluid 101 along the conduit 139 and expelling the fluid via dispensing aperture 142 for use. The pump 145 can be a peristaltic pump. The pump 145 can be used to exert pressure on a pod to drive thicker, heavier creams/fluids from the pod 140.

FIGS. 12D and 12E each use a plunger 171 to drive fluid 101 from the pod 140. FIG. 12D urges the pod 140 against a piercing tip 166 to puncture the pod 140 at the time of use and release fluid 101. This will keep the fluid fresh 101 until ready for dispensing and use. In contrast FIG. 12E uses a syringe mechanism, with a constantly open outlet orifice 104. This syringe style can be suitable for thicker fluids 101, however, thin, more viscous fluids can leak through the outlet orifice 104 without plunger 171 application.

Finally, FIG. 12F illustrates a reverse syringe where the fluid 101 is dispensed contra to the direction of the plunger 171 movement, black arrows indicating the direction of plunger 171 movement, and light colour arrows indicating the direction of flow of fluid 101.

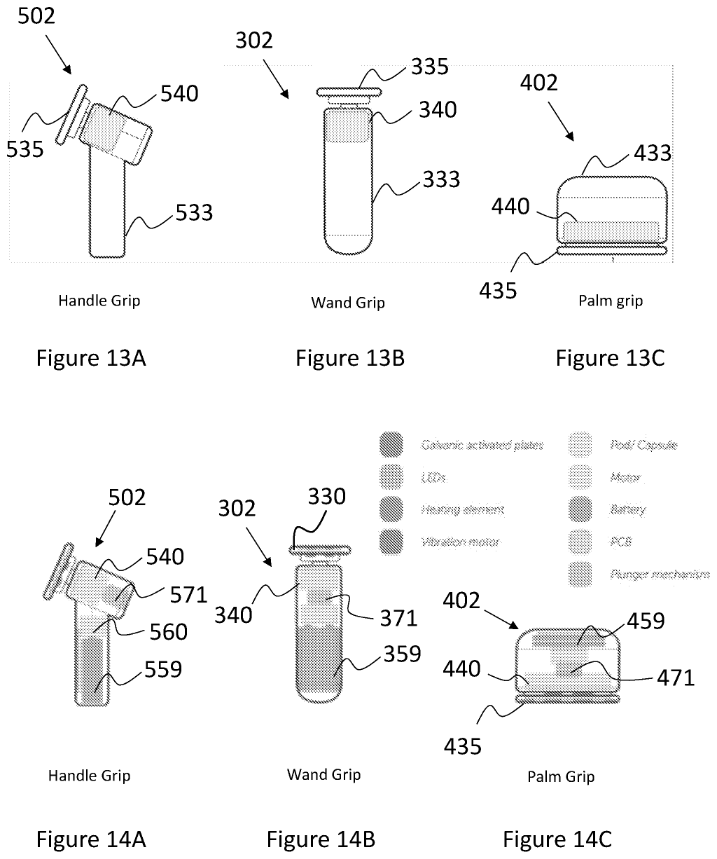

Further to contemplating various pod 140 configurations, it is also contemplated that the skin care device 102 can be manufactured in different shapes and forms. FIG. 13A illustrates a “handle-grip” embodiment of the device 502 having a head 535 and a handle 533 offset thereto. This is contrasted to FIG. 13B where skin care device 302 has a “wand grip” providing a head 335 in line with a handle 333. FIG. 13C illustrates a “palm-grip” embodiment of the skin care device 401 where a head 435 is disposed on a front face of a block-shaped handle 433. Each of the devices 302, 402, 502 is openable to receive and replace pods 340, 440, 540 therein. To accommodate the different handles 333, 433, 533 the internal packaging of the devices is reconfigured. As illustrated in FIGS. 14A-14C, each device contains the following components in a head: galvanic plates 329, 429, 529; LEDs 330, 430, 530; heating element 334, 434, 535; and vibration motor 360,460,560. Additionally, each device contains the following components in a body: pod 340, 440, 540; motor 360, 460, 560; battery 359, 459, 559; printed circuit board (PCB) 361, 461, 561; and plunger mechanism 371, 471, 571.

The second embodiment of the skin care device 302 is shown in more detail in FIGS. 15A and 15B, having a slim line handle 333 and linear internal layout. The head 335 is angled slightly in relation to the handle 333 for ergonomic considerations in that the user will be more comfortable when holding the galvanic plates 329 of the contact surface 337 to their face for extended periods of time.

The handle 333 is made from first 333a and second 333b portions each having a semi-circular cross-section such that on being interconnected a cylindrical cavity is formed in the handle 333 for housing the required internal componentry of the device 302.

FIG. 15A illustrates the contact surface 337 which can be heated by a heat element 334 located directed under the contact surface 337.

Disposed equidistantly around a periphery of the contact surface 337, in a trilobal configuration, is a plurality of galvanic elements 329. These elements 329 when activated generate an electric filed to deliver a current and/or voltage to the user's skin to produce the iontophoresis effect described herein. Centrally located upon the contact surface 337 is a fluid dispensing aperture 342 for expelling the fluid 101 from the pod 340. It is contemplated that there can be a plurality of dispensing apertures 342 (although not illustrated) to more evenly spread the fluid 101 across the contact surface 337. Alternatively, the plurality of apertures 342 can be configured to expel differed fluids 101 to be used independently or in conjunction with one another over the course of a treatment cycle.

Peripherally disposed around the head 334 is the LED array 330, which when activated can deliver a plurality of therapeutic light wave lengths to the skin of the user, for treatment and/or therapy. At an upper end of the handle 333 immediately adjacent the head 335 is a gripping portion 368 made from a soft or silicon rubber to provide an increase friction portion of the handle for improved grip. The gripping portion 368 can be extended around/across any portion of the head 335 and/or the handle 333 for improved grip and protection of the device 302 if accidentally dropped. Where fluid 101 from the aperture 142 is transferred to the handle 333 or user's hand a hard plastic handle 333 can become slippery. To this end, the entire handle 333 can be made from a soft-touch plastic or silicon, or coated with a sleeve of high friction or soft touch material.

On a rear of the device 302 is an array of buttons 150. The button array 150 is linearly arranged along the handle 333, with each button etched or embossed or otherwise marked to indicate the operative function thereof.

A prominent, large button at a base of the handle 333 is the on/off button 151, used to awaken and activate the device 302. Above the on/off button 151 an LED indicator 152 shows the level of charge in the device 302 to alert the user to when recharging or battery replacement of the device 302 is required.

The remaining buttons of the array 350 are directed to activating various functions of the device 302 and are shown in FIG. 15B in but one of a number of possible configurations. Button 353 can activate the heating element 334 under the contact surface 337. In some embodiments the heating element can also be configured to cool an area of the skin in contact with the contact surface 337. Button 354 can initiate iontophoresis by activating a current in the galvanic plates 329. Button 355 can activate the LED lights 330, with each successive push of the button 355 cycling through red, green and blue light treatment settings. Button 356 can activate the vibration motor 362 to provide a massaging effect to the skin in combination with the other described treatments. Button 357 is configured as a flow rate selector positioned adjacent to a plurality of LED indicators, indicating to the user whether the dispensing flow rate is high, medium or low. Finally, an elongate button 358 initiates dispensing of the fluid to begin an infusion process into the user's skin.

It is contemplated that some of the independent functions of the device 302 can be incorporated into a treatment routine by the control processor 146 as described herein. For example, a skin analysis result may indicate that the user's skin is dehydrated, and in response the control processor 146 can activate a hydration routine encompassing a treatment fluid, a heat treatment and a light treatment. This arrangement could negate the need for individual buttons to activate the varying functionalities of the device. Additional buttons can be incorporated for a camera to take images of the skin or to initiate skin assessment analysis.

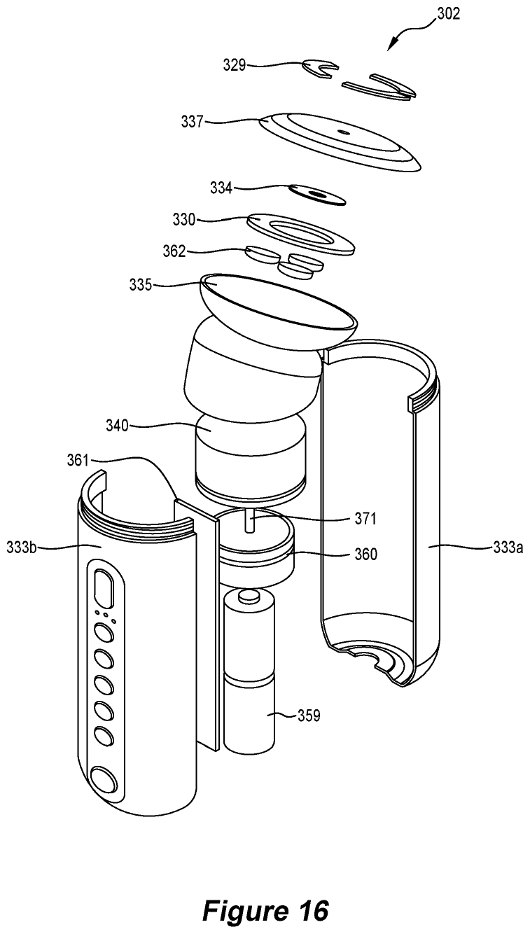

An exploded view of the skin care device 302 in FIG. 16 illustrates the internal layout of components therein. The two handle portions 333a, 333b are separated revealing a pair of AA batteries (359) in series, and aligned with a PCB 361. Adjacent the batteries 359 is the motor 360 configured to drive the plunger mechanism 371 and urge the fluid 101 from the pod 340.

The plunger mechanism 371 applies pressure to a diaphragm or evacuator 164 of the pod 340 forcing fluid 101 therein out of the pod outlet orifice 304. The orifice 304 is in fluid communication with the fluid dispensing aperture 342 of the device 302 to deliver the fluid directly to the user's face.

The pod outlet orifice 304 delivers the fluid 101 to the head 335 of the device 302 to be dispersed across the contact surface 337 via aperture 342. Below the contact surface 337 is a circular array of LEDs 330 for providing a light source for light treatment. Additionally, there is a secondary, vibration motor 362 for oscillating the control surface 337 and components thereon eg. the galvanic plates 329. The vibrating motor 362 can be in the form of an eccentric rotating mass vibration motor (ERM) which uses a small offset mass on a direct current motor to induce a translational force to each rotation of the motor. Alternatively, the motor 362 can house a linear resonant actuator (LRA) a basic spring mass system that will induce a force to produce oscillatory motion in the motor.

Also nestled below the control surface 337 is the heating/cooling element 334 for adjusting the temperature of the control surface 337 whether this is heating or cooling.

As described in relation to FIG. 12C the internal arrangement of FIG. 16 can be rearranged to position a peristaltic pump 345 either below or above the pod 340, to pump fluid 101 therefrom. The relative positions of the pump 345, the pod 340 and the conduit 339 are tailored to the split line that divide the handle 333 into portions 333a and 333b for ease of replacement and removal of the pod 340 within the device 302.

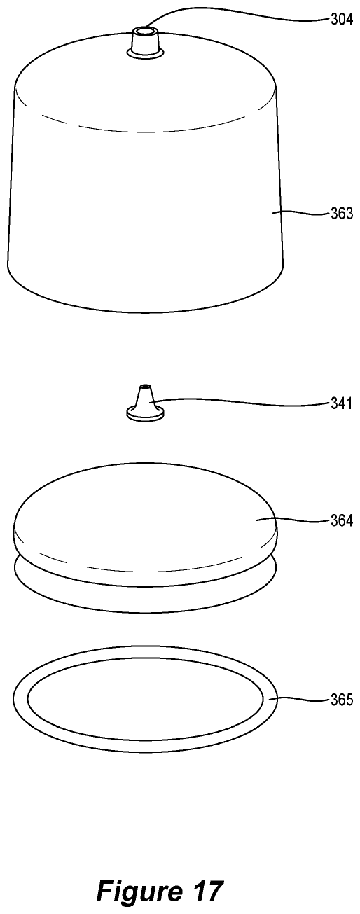

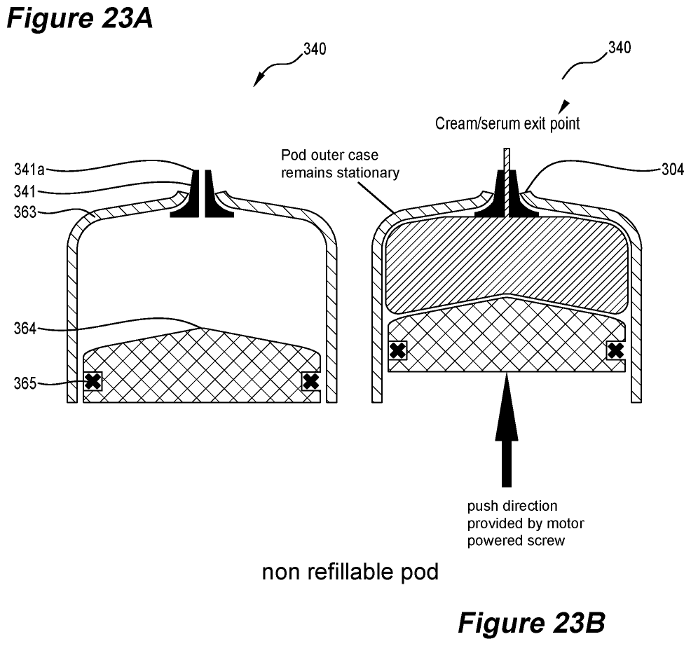

The pod 340 is illustrated in more detail in the exploded view of FIG. 17. The pod 340 comprises an outer shell or casing 363 containing the pod outlet orifice 304. The casing 363 can be rigid, semi-rigid or soft dependent on the expulsion method of the device 302. For example, a soft casing 363 will be easier and require less force to squeeze a fluid out from. In comparison a rigid casing 363 may be suitable where the pump 345 is being used to expel the fluid and compression of the pod casing 363 is not required. A rigid casing 363 can be more hard wearing and lend itself to a refillable pod. In another form, the rigid casing 363 can be employed with an inflexible evacuator 364 (as illustrated in FIGS. 23A-23B) to be refillable by the user.

Inside the casing 363 immediately adjacent the orifice 304 is a valve illustrated in FIG. 17 as a duckbill valve 341 to control the flow and pressure of the fluid 101 exiting the pod 340. The diaphragm or evacuator 364 is located in an opening of the casing 363 and sealed thereto via a seal 365. The evacuator 364 can be solid, and, in use, is plunged in combination with the seal 365 into the casing 363 until the fluid 101 is depleted. In some embodiments, the evacuator is a flexible diaphragm 364 that does not move in relation to the casing 363. It is instead pushed and deformed into the casing by a plunger mechanism 371 until it conforms to the size and profile of the casing 363 such that the fluid 101 is total expelled from the casing 363.

In some embodiments, the valve 341 can be integrally formed with the pod casing 363. In some embodiments the valve 341 is formed independently of the casing, and inserted into the orifice 304, to be held in place by the internal pressure of the fluid 101 driving a base of the valve 341 directly up against the casing 363 in proximity to the orifice 304. The overall dimensions of the pod 340 are such that the pod 340 houses in its entirety within the handle 333 of the device 302, and can be removed and replaced within the handle 333 by loosening or reorienting of the head 335 of the device 302.

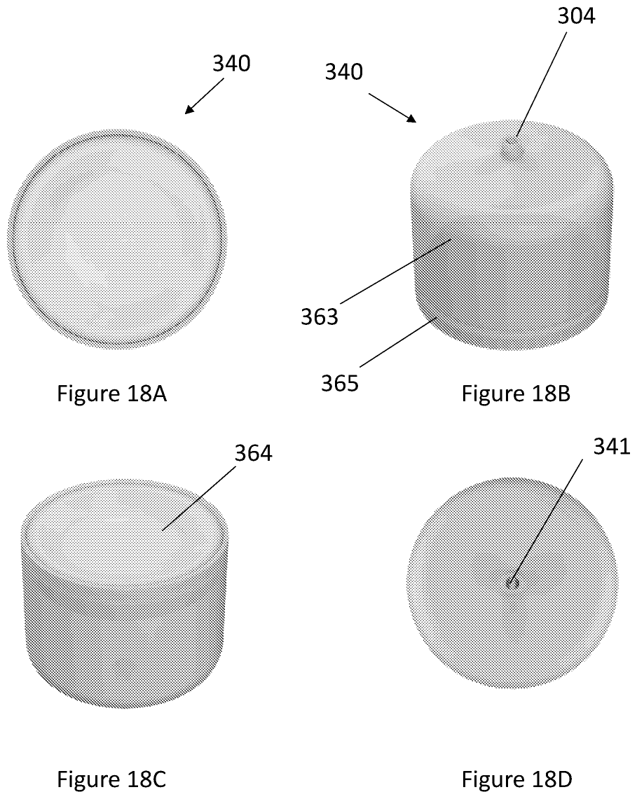

FIGS. 18A and 18C illustrate a bottom of the pod 340 and a concave shape to the diaphragm 364 that curves into the casing 363 of the pod 340.

FIG. 18B is a top perspective view of the pod 340 with the casing 363 semi-transparent to show the diaphragm 364 therein. The orifice 304 in use will be fluidly connected to a conduit 359 to deliver the fluid to the aperture 342 on the contact surface 337. The bottom perspective view of FIG. 18C also illustrates the concave of the diaphragm creating a recess in a base of the pod 340 for receiving the plunger mechanism 371.

FIG. 18D is a top view of the pod 340 and displays the valve 341 sitting centrally of the orifice 304 to control the flow of fluid 101 from the pod 340 and to reduce the risk of the fluid clogging or solidifying around the orifice 304.

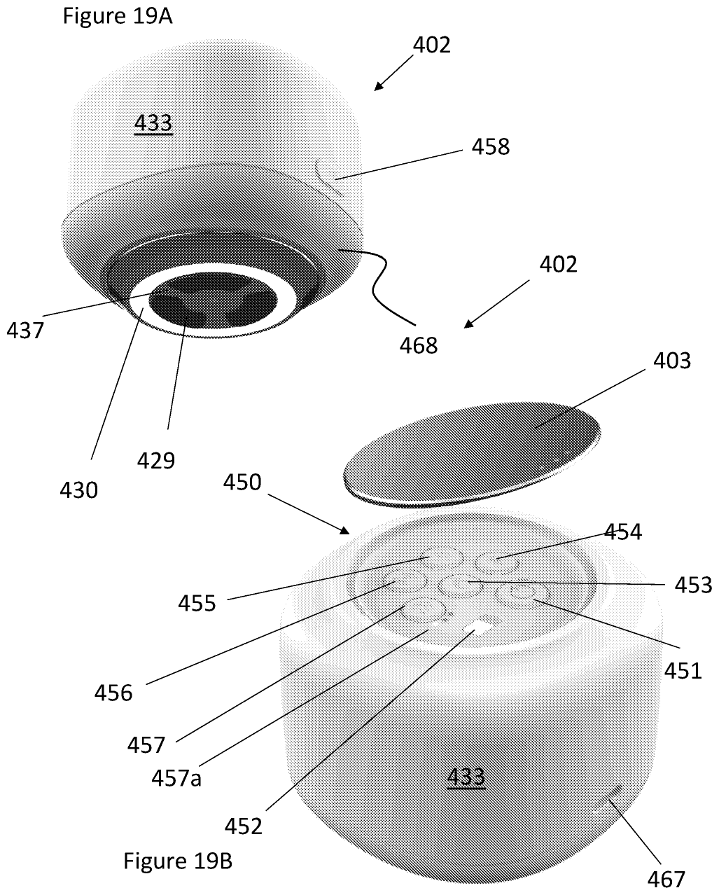

A third exemplary skin care device 402 is illustrated in FIGS. 19A and 19B showing bottom and top perspective views of the skin care device 402. The device 402 is configured to be held in a palm of the user's hand and utilises a more compact package of components therein in comparison to device 302. A removable lid 403 covers the button array 450 and is held in place by a hinge or a snap-fit configuration or by magnetism. The user can select the buttons to program the treatment required before replacing the lid 403 before beginning use.

The buttons 450 provide an on/off button 451 to activate the device and a battery level indicator 452 with similar function to those described herein in relation to device 302. Additionally, button 453 can activate a heating element 434 under a contact surface 437. Alternatively, the heating element can be configured to reduce heat or cool an area of the skin in contact with the contact surface 437. Button 454 can initiate iontophoresis by activating an electrical current in a galvanic plate 429. Button 455 can activate an LED light array 430, with each successive push of the button 455 cycling through red, yellow, green and blue light treatment settings. Button 456 can activate a vibration motor 462 to provide a massaging or numbing effect to the skin in combination with the other described treatments. Button 457 is configured as a flow rate selector positioned adjacent to a plurality of pin-point LED indicators 457a, indicating to the user whether the dispensing flow rate is high, medium or low. Finally, an elongate button 458 located on the handle or body 433 of the device 402 can initiate dispensing of the fluid or initiation of a treatment, to begin an infusion process into the user's skin for the treatment. A recharge interface or port 467 is also located in the body 333 to enable recharging of internal batteries.

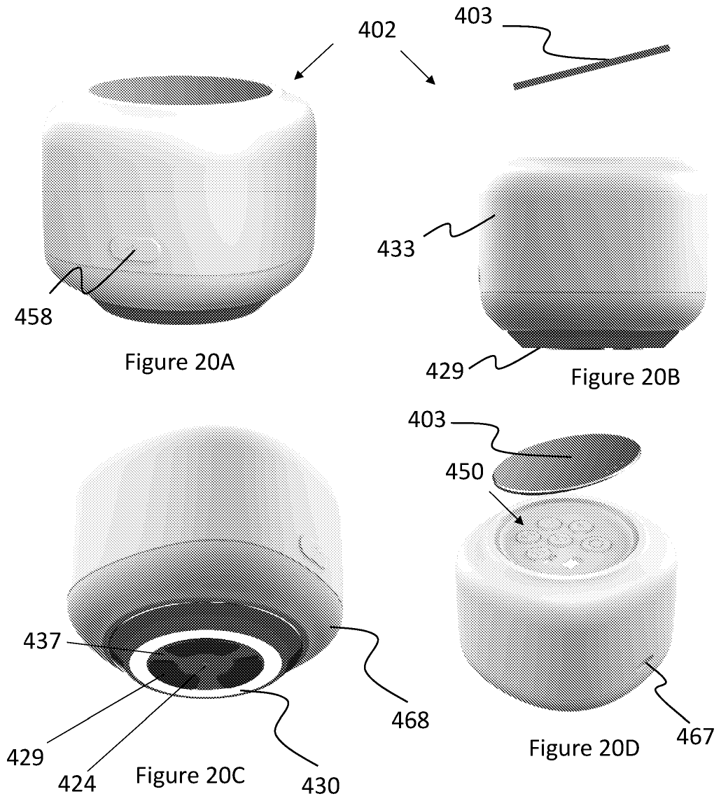

FIGS. 20A-20D illustrate a perspective, an open-side, a bottom perspective and an open-top perspective view of the skin care device of FIGS. 19A and 19B, respectively. A collar 468 is disposed around the head 435 to assist the user in gripping the device 402. The collar can be a textured material or a rubberised or silicon material that the user rests their fingers upon with the body 433 of the device 402 in their palm. The device 402 is then held in contact with the user's skin around the face or neck or alternative skin surface to be treated, as the treatment cycle is executed. The skin surfaces to be treated can be confined to the face and neck but may also be extended to the décolletage, arms, shoulders, legs, stomach depending on the skin treatment requirements of the user.

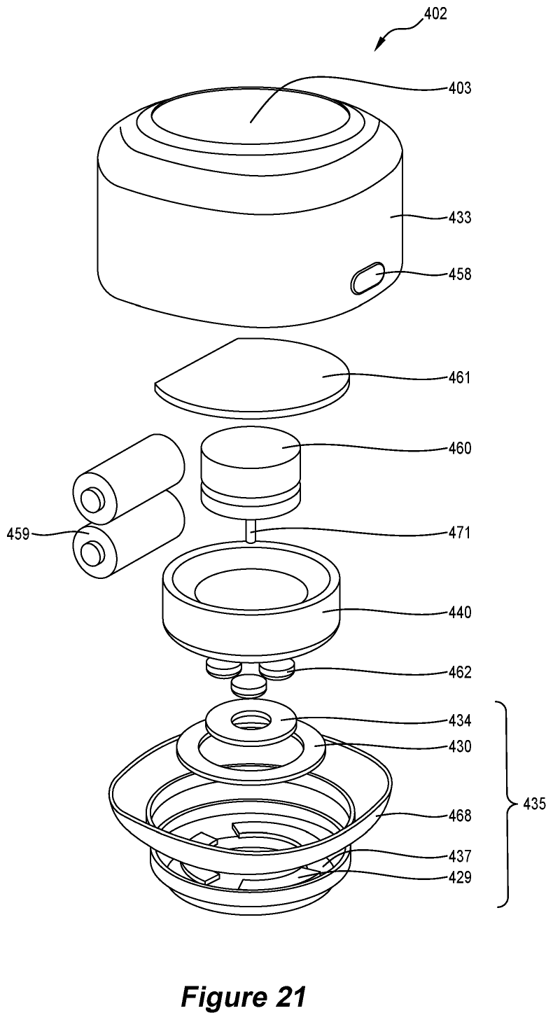

The packaging of the internal components of device 402 are illustrated in the exploded view of FIG. 21. Inside the body 433 is a planar, laminate printed circuit board 461 that abuts the button array 450. Adjacent the PCB 461 is a motor 460 configured to drive a plunger mechanism 471. In some embodiments of the device 402 the plunger mechanism 471 can be substituted for a pump or peristaltic pump to draw the fluid 101 from the pod 440 as opposed to deforming or squeezing the pod 440 to expel the fluid 101.

The plunger mechanism 471 is in direct contact with the pod 440, and preferably the diaphragm 464 at the base of the pod 440 to deform the diaphragm forcing fluid into either a conduit 439 or directly to the dispensing aperture 442 on the contact surface 437 of the device 402. Also contained within the body 433 is a power source, illustrated as a pair of batteries 459. These can be standard AA batteries or rechargeable batteries. The batteries 459 can also be substituted for a rechargeable power pack (not illustrated).

Below the contact surface 437 is a similar stack of coaxially aligned components as that of device 302. Namely, a vibrating motor or array of vibrating motors 462; a heating element 434; and a circular LED array 430 configured to provide at least one of red, green or blue light.

The head 435 is formed of a base element configured as the contact surface 437 upon which a plurality of galvanic plates 429 are equidistantly disposed, and a central aperture 442 for dispensing the fluid 101 therefrom.

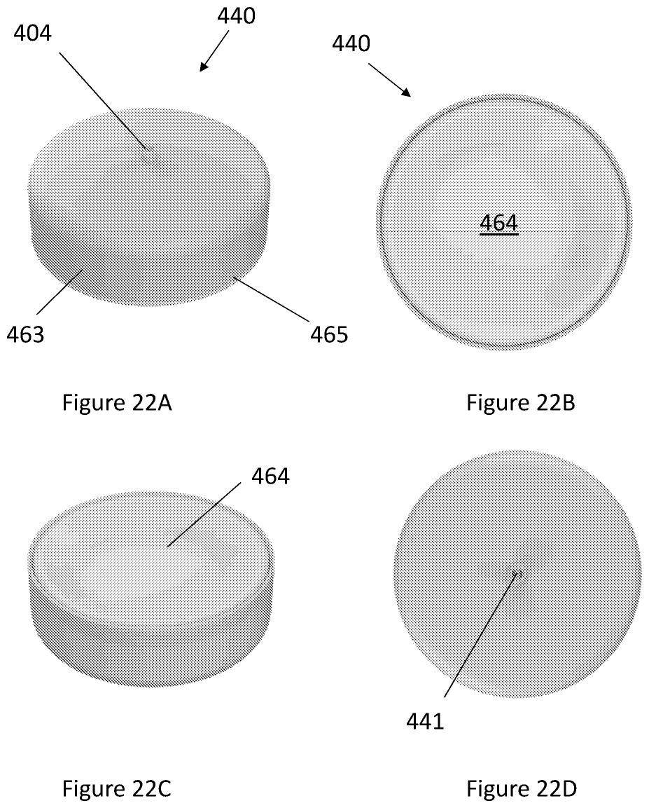

FIGS. 22A-22D illustrate a top perspective, a bottom, a bottom perspective and a top view of the pod 440 of FIG. 21, respectively. The pod 440 is shallower with a larger cross-section than that of pod 340, which is configured to reside within the narrower, slimline handle 333 of the device 302. However, the features of pod 440 are the same as those described in relation to pod 340. A casing 463 provides a cavity for holding fluid 101. The casing 463 is sealed by a pod evacuator or diaphragm 464. Centrally of the casing 463 is a pod outlet orifice 404 in which a valve 441 is positioned. The valve 441 can be a duck bill valve and provides control of the fluid 101 as it is dispensed. A peripheral seal 465 maintains the diaphragm 464 in close contact with the casing 463 to prevent seepage of the fluid 101 therefrom.

FIGS. 23A and 23B illustrate two cross-sectional views of the pod 340 illustrating, by arrow, a direction of movement of the diaphragm 364 that forces the fluid 101 out of the pod 340 on demand. The pod 340 illustrated in FIG. 23B has a rigid casing 363 and the diaphragm 364 is rigid, essentially an evacuator, that is forced by the plunger mechanism 371 into the casing 363 sliding the seals 365 further into the casing on each use to expel the fluid 101 through the duck bill valve 341 and out of a valve aperture 341a. The fluid 101 can then be expelled directly to the aperture 342 in the head 335 of the device 302, or forced along a conduit 359 to subsequently be expelled from the aperture 342 in the head of the device.

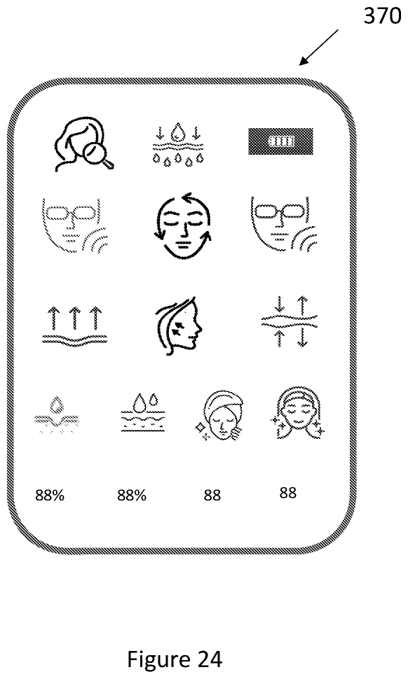

FIG. 24 is an exemplary display 370 that can be available from the device 102,302,402,502 itself, or from a paired user device, illustrating available features and skin analysis results to the user. The display 370 will indicate which elements of the device are active during a treatment and also displays the skin analysis in a percentage or similar easy to read form, where 100% is a perfect result. The display 370 can be coloured or otherwise illuminated to provide additional information to the user including a battery level indicator.

### Conclusion

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

LEGEND

| #Ref | Title | # Ref | Title |
|---|---|---|---|
| 101 | Fluid (cream, | 144 | Plasma jet control |
| | lotion, medicament) | | |
| 102 | Skin care device | 145 | Pump |
| 103 | Lid | 146 | Control processor |
| 104 | Pod outlet orifice | 148 | User interface of device |
| 108 | Application | 150 | Buttons |
| 206 | Partition | 151 | Power button |
| 208 | Actuator of pod | 152 | Battery level indicator |
| 124 | Sensors | 153 | Heat activation button |
| 126 | Camera | 154 | Iontophoresis button |
| 128 | Camera light | 155 | Light activation button |
| 129 | Galvanic plate | 156 | Vibration activation button |
| 130 | LEDs | 157 | Flow rate selector |
| 131 | Vibration pad | 158 | Infuse [dispense] button |
| 132 | Vibration actuator | 159 | Battery |
| 133 | Handle | 160 | Motor |
| 134 | Heat element | 161 | PCB |
| 135 | Head | 162 | Vibration motor |
| 136 | Iontophoresis module | 163 | Pod casing |
| 137 | Contact surface | 164 | Diaphragm (Pod evacuator) |
| 138 | Fluid dispenser module | 165 | Pod seal |
| 139 | Fluid conduit of device | 166 | Tip |
| 140 | Fluid container | 167 | Recharge interface |
| 141 | Valve | 168 | Grip portion |
| 141a | Valve aperture | 169 | Charging base |
| 142 | Fluid dispensing | 170 | Display |
| | aperture/s of device | | |
| 143 | Fluid pump | 171 | Plunger mechanism |
| | | 600 | User interface |
| 300 | Application face | 700 | User interface |
| 400 | Dashboard interface | 800 | User interface |
| 500 | Analysis interface | 900 | Skin quality report |
| | | 901 | Skin image wavelength 1 |
| | | 902 | Skin image wavelength 2 |
| | | 903 | Moisture analysis chart |
| | | 904 | Expert advice |
| | | 905 | Recommendation/info |

1. A skin care apparatus having a body, a head and a control processor,
the head comprising:
a user contact surface;
a light device for emitting light at 455 nm, 633 nm, and 830 nm;
a vibration pad;
a heating element;
a skin moisture sensor; and
an iontophoresis module including at least one plate member mounted on the user contact surface,
each disposed around the head for placing in proximity to a user's skin, the body housing:
a fluid container configured to store at least one skin care-related fluid; and
a dispensing module for controlled dispensing of the skin care-related fluid from the fluid container to the head of the apparatus, and
the control processor configured to:
operate the skin moisture sensor to perform a skin quality assessment of the user's skin; and
operate any one of more of the light device, the vibration pad, the heating element, and the iontophoresis module, in combination with the dispensing module to provide a bespoke skin treatment for the user's skin based on the skin quality assessment.

2. The apparatus of claim 1, further comprising a transceiver for communication with an application on a user device, wherein the control processor is configured to operate in connection with the transceiver to:
transmit data from the skin moisture sensor to the application for performing the skin quality assessment; and
receive instructions from the application for operating any one or more of the light device, the vibration pad, the heating element, and the iontophoresis module to provide the skin treatment.

3. The apparatus of claim 1, wherein the skin care apparatus further comprises a pH level sensor configured to be operated by the control processor to determine a pH level for the user's skin.

4. The apparatus of claim 1, wherein the application is configured to use the data from at least one of the skin moisture sensor and the pH level sensor to determine skin quality parameters for the user's skin including at least one of, a moisture/dryness level, a detection of wrinkles/lesions, and a detection of acne.

5. The apparatus of claim 1, wherein the head further comprises a camera configured to be operated by the user and/or the control processor for capturing and transmitting visual images of the user's skin to perform the skin quality assessment.

6. The apparatus of claim 5, wherein the application is configured to use one or more images from the camera to determine skin quality parameters for the user's skin.

7. The apparatus of claim 1, wherein the dispensing module is configured to be activated by the user to communicate fluid to the head of the apparatus before and/or during operation of any one of more of the light device, the vibration pad, the heating element, and the iontophoresis module to promote transdermal delivery of the fluid.

8. The apparatus of claim 1, wherein the dispensing module is configured to be activated by the control processor to communicate skin care-related fluid to the head of the apparatus before and/or during operation of any one of more of the light device, the vibration pad, the heating element, and the iontophoresis module to promote transdermal delivery of the fluid.

9. The apparatus of claim 1, wherein the application is configured to determine and display a recommendation based on the skin quality parameters, the recommendation including application of at least one skin care-related fluid.

10. The apparatus of claim 9, wherein the application is configured to determine instructions for operating the light device, the vibration pad, the heating element, and the iontophoresis module based on at least one of the recommendation and the skin quality parameters.

11. The apparatus of claim 10, wherein the recommendation is selectable and selection of the recommendation causes the application to transmit the instructions to the control processor.

12. The apparatus of claim 11, wherein the application communicates with the apparatus to track when recommendations are selected to determine when the recommendations are to be modified.

13. The apparatus of claim 10, wherein the application is configured to specify in the instructions that a red LED of the light device is to be activated for a predetermined duration per skin treatment for skin quality parameters corresponding to an anti-aging treatment or a skin sensitivity treatment.

14. The apparatus of claim 10, wherein the application is configured to specify in the instructions that a blue LED of the light device is to be activated for a predetermined duration per skin treatment for skin quality parameters corresponding to an acne control treatment.

15. The apparatus of claim 1, wherein the light device includes at least one LED that emits blue light at 455 nm, at least one LED that emits red light at 633 nm, and at least one LED that emits infrared light at 830 nm to promote skin healing.

16. The apparatus of claim 1, wherein the vibration pad includes a vibration actuator configured to vibrate at a frequency to numb the user's skin during the skin treatment.

17. The apparatus of claim 16, wherein the vibration actuator is configured to vibrate at 7000 RPM to provide acoustic waves at the frequency to numb the user's skin.

18. The apparatus of claim 1, wherein the heating element includes a radio transmitter configured to provide localised tissue heating via radiofrequencies between 100 kHz-300 GHz.

19. The apparatus of claim 1, wherein the iontophoresis module is configured to provide a voltage gradient to the user's skin through creation of an electric field.

20. The apparatus of claim 1, wherein the dispensing module comprises:
a pump or actuator to drive a volume of skin care-related fluid from the fluid container;
a conduit for delivering the skin care-related fluid from the fluid container to the head of the apparatus; and
an outlet to dispense the volume of skin care-related fluid from the head to the user's skin.

21. The apparatus of claim 20, wherein a portion of the fluid container is selectively deformed or displaced when the dispensing module is activated by the user to dispense skin care-related fluid from the head.

22. The apparatus of claim 21, wherein the fluid container is removable and selected from a plurality of fluid containers based on a type of the skin treatment.

23. The apparatus of claim 1, further comprising a user interface including a sense button, wherein activation of the sense button causes the skin moisture sensor to detect data including at least one of a skin moisture level, and a skin dryness level of the user's skin.

24. The apparatus of claim 1, wherein the head further comprises a skin heat sensor to measure the temperature of the user's skin.

25. The apparatus of claim 1, wherein the head further comprises at least one of a pressure sensor, a biometric sensor, and a conductivity sensor to detect when the head of the apparatus is placed adjacent or in contact with the user's skin.

26. The apparatus of claim 1, further comprising a user interface including a capture button, wherein activation of the capture button causes a camera to record an image of the user's skin.

27. A fluid container configured to be removably housed within the apparatus of claim 1, the fluid container comprising:
a casing;
an evacuator; and
a valve for controlling a dispensing rate of skin care-related fluid from an aperture of the fluid container,
wherein at least one of the user and the instructions received from the application initiate dispensing of a volume of skin care-related fluid to the head of the apparatus in combination with provision of the bespoke skin treatment.

28. A skin care apparatus having a body, a head and a control processor,
the head comprising:
a user contact surface;
an iontophoresis module including at least one plate member disposed upon the user contact surface to supply at least one of electrical current and voltage;
a skin care-related fluid dispensing outlet located on the user contact surface;
a skin moisture sensor;
a heating element to supply heat to a user's skin;
a vibration pad to vibrate the head and the at least one plate member provided thereon; and
a light device disposed below the user contact surface, the light device comprising a plurality of LEDs for emitting light at 455 nm, 633 nm, and 830 nm to a user's skin, the body housing:
a fluid container configured to store at least one skin care-related fluid; and
a dispensing module for controlled dispensing of the skin care-related fluid from the fluid container to the skin care-related fluid dispensing outlet on the head of the apparatus,
wherein the control processor is configured to:
operate the skin moisture sensor to perform a skin quality assessment of the user's skin; and
operate any one of more of the light device, the vibration pad, the heating element, and the iontophoresis module, in combination with the dispensing module to provide a bespoke skin treatment based on the skin quality assessment of the user's skin.

29. The skin care apparatus of claim 28, wherein the at least one plate member is a galvanic plate.

30. The skin care apparatus of claim 29, wherein the iontophoresis module comprises a plurality of galvanic plates disposed around a periphery of the user contact surface.

31. The skin care apparatus of claim 28, wherein the skin moisture sensor is configured to use the at least one plate member to measure skin quality.

32. The skin care apparatus of claim 28, wherein the heating element supplies heat to the user's skin via the at least one plate member or the user contact surface.

33. The skin care apparatus of claim 28, wherein the plurality of LEDs are configured to selectively emit light at 455 nm, 633 nm, and 830 nm through the user contact surface in combination with the supply of at least one of current and voltage via the at least one plate member to the user's skin.

34. The skin care apparatus of claim 28, wherein the at least one plate member is configured to supply current and voltage to the user's skin simultaneously to facilitate absorption of the skin care-related fluid.

35. The skin care apparatus of claim 33, wherein the at least one plate member is configured to supply at least one of current and voltage to the user's skin to facilitate absorption of the skin care-related fluid in combination with the vibration pad vibrating the head.

36. The skin care apparatus of claim 28, wherein the vibration pad uses a linear resonating actuator (LRA) mounted within the head to effect vibration of the head and the at least one plate member to induce vibration of the user's skin.

37. The skin care apparatus of claim 28, wherein the vibration pad uses an eccentric rotating mass vibration motor (ERM) mounted within the head to effect vibration of the head and the at least one plate member to induce vibration of the user's skin.
